(19) United States
(12) Patent Application Publication
(10) Pub. No.: US 2016/0324557 A1
(43) Pub. Date: Nov. 10, 2016

(54) SURGICAL IMPLANT SYSTEM AND METHOD OF USE

(71) Applicant: Warsaw Orthopedic, Inc., Warsaw, IN (US)

(72) Inventors: Langston Holly, Encino, CA (US); Mark C. Dace, Collierville, TN (US)

(21) Appl. No.: 14/705,747

(22) Filed: May 6, 2015

(51) Int. Cl. A61B 17/86; A61B 17/70

(52) U.S. Cl. CPC A61B 17/863; A61B 17/7059; A61B 17/8635

(57) ABSTRACT

A spinal implant includes a proximal end including a thread and at least one resistance element. A distal end includes the thread and is configured to penetrate tissue. The at least one resistance element is engageable to increase a resistance during penetration of the tissue. Systems and methods of use are disclosed.

### TECHNICAL FIELD

The present disclosure generally relates to medical devices for the treatment of spinal disorders, and more particularly to a surgical implant system including a bone fastener and a method for treating a spine.

### BACKGROUND

Spinal pathologies and disorders such as scoliosis and other curvature abnormalities, kyphosis, degenerative disc disease, disc herniation, osteoporosis, spondylolisthesis, stenosis, tumor, and fracture may result from factors including trauma, disease and degenerative conditions caused by injury and aging. Spinal disorders typically result in symptoms including deformity, pain, nerve damage, and partial or complete loss of mobility.

Non-surgical treatments, such as medication, rehabilitation and exercise can be effective, however, may fail to relieve the symptoms associated with these disorders. Surgical treatment of these spinal disorders includes correction, fusion, fixation, discectomy, laminectomy and implantable prosthetics. As part of these surgical treatments, implants such as bone fasteners, plates, connectors and vertebral rods are often used to provide stability to a treated region. These implants can redirect stresses away from a damaged or defective region while healing takes place to restore proper alignment and generally support the vertebral members. For example, plates may be attached via the fasteners to the exterior of one or more vertebral members. This disclosure describes an improvement over these prior art technologies.

### SUMMARY

In one embodiment, a spinal implant for use with a surgical treatment is provided. The spinal implant comprises a proximal end including a thread and at least one resistance element. A distal end includes the thread and is configured to penetrate tissue. The at least one resistance element is engageable to increase resistance during penetration of the tissue. In some embodiments, systems and methods are disclosed.

### BRIEF DESCRIPTION OF THE DRAWINGS

The present disclosure will become more readily apparent from the specific description accompanied by the following drawings, in which:

FIG. 1 is a side view of one embodiment of components of a system in accordance with the principles of the present disclosure;

FIG. 2 is a break away view of the components shown in FIG. 1;

FIG. 3 is a side view of one embodiment of components of a system in accordance with the principles of the present disclosure;

FIG. 4 is a perspective view of one embodiment of components of a system in accordance with the principles of the present disclosure disposed with vertebrae; and

FIG. 5 is a side view of one embodiment of components of a system in accordance with the principles of the present disclosure.

### DETAILED DESCRIPTION

The exemplary embodiments of a surgical system and related methods of use disclosed are discussed in terms of medical devices for the treatment of musculoskeletal disorders and more particularly, in terms of a spinal implant system including a bone fastener and a method for treating a spine.

In one embodiment, a spinal implant, such as, for example, a bone screw is configured for insertion into a spinal implant, such as, for example, a spinal plate and/or into tissue, such as, for example, bone. In one embodiment, the bone screw provides a tactile feel to the surgeon when the screw is about to be fully inserted thereby avoiding over-screwing and stripping of bone.

In one embodiment, a spinal implant, such as, for example, a bone screw is provided having a tactile indicator configured to indicate that the bone screw is approaching a final position. In some embodiments, the tactile indicator allows bone screws to be inserted into a patient while a head of the bone screw is obscured by instrumentation or patient anatomy. In some embodiments, the tactile indicator facilitates insertion and determination that the bone screw is fully-seated when there is an obstruction of the bone screw thereby preventing the bone screw from being over-driven causing the bone screw purchase to be reduced.

In some embodiments, the bone screw having the tactile indicator is utilized with an implant, such as, for example, a cervical plate. In one embodiment, the bone screw includes a shaft having a full-length thread and an additional thread. In one embodiment, the additional thread is positioned near a neck portion of the bone screw to increase an insertion torque when the additional thread makes contact with the plate. In one embodiment, the tactile sensation of the increased torque indicates that the bone screw is substantially fully-seated. In one embodiment, the additional thread includes the same pitch as the full-length thread.

In some embodiments, the tactile indicator may include, such as, for example, one or more ribs, spikes, bosses, or protrusions that interact with the surface of tissue to increase the insertion torque to achieve the tactile indication. In some embodiments, the bone screw includes the tactile indicator at a proximal end of the screw to generate the tactile feedback and indicate the proper stage of insertion.

In some embodiments, the present disclosure may be employed to treat spinal disorders such as, for example, degenerative disc disease, disc herniation, osteoporosis, spondylolisthesis, stenosis, scoliosis and other curvature abnormalities, kyphosis, tumor and fractures. In some embodiments, the present disclosure may be employed with other osteal and bone related applications, including those associated with diagnostics and therapeutics. In some embodiments, the disclosed spinal implant system may be alternatively employed in a surgical treatment with a patient in a prone or supine position, and/or employ various surgical approaches to the spine, including anterior, posterior, posterior mid-line, direct lateral, postero-lateral, and/or antero lateral approaches, and in other body regions. The present disclosure may also be alternatively employed with procedures for treating the lumbar, cervical, thoracic, sacral and pelvic regions of a spinal column. The spinal implant system of the present disclosure may also be used on animals, bone models and other non-living substrates, such as, for example, in training, testing and demonstration.

The present disclosure may be understood more readily by reference to the following detailed description of the embodiments taken in connection with the accompanying drawing figures, which form a part of this disclosure. It is to be understood that this application is not limited to the specific devices, methods, conditions or parameters described and/or shown herein, and that the terminology used herein is for the purpose of describing particular embodiments by way of example only and is not intended to be limiting. Also, in some embodiments, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment. It is also understood that all spatial references, such as, for example, horizontal, vertical, top, upper, lower, bottom, left and right, are for illustrative purposes only and can be varied within the scope of the disclosure. For example, the references “upper” and “lower” are relative and used only in the context to the other, and are not necessarily “superior” and “inferior”.

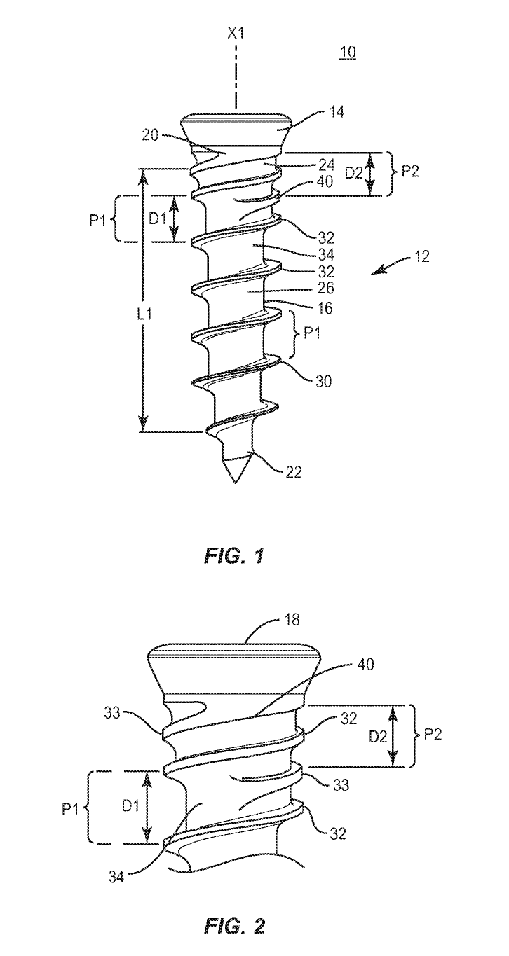

The following discussion includes a description of a surgical system including a bone screw, related components and methods of employing the surgical system in accordance with the principles of the present disclosure. Alternate embodiments are also disclosed. Reference will now be made in detail to the exemplary embodiments of the present disclosure, which are illustrated in the accompanying figures. Turning to FIGS. 1 and 2, there are illustrated components of a spinal implant system 10 including a spinal implant, such as, for example, a bone screw 12.

The components of spinal implant system 10 can be fabricated from biologically acceptable materials suitable for medical applications, including metals, synthetic polymers, ceramics and bone material and/or their composites. For example, the components of spinal implant system 10, individually or collectively, can be fabricated from materials such as stainless steel alloys, commercially pure titanium, titanium alloys, Grade 5 titanium, super-elastic titanium alloys, cobalt-chrome alloys, stainless steel alloys, superelastic metallic alloys (e.g., Nitinol, super elasto-plastic metals, such as GUM METAL® manufactured by Toyota Material Incorporated of Japan), ceramics and composites thereof such as calcium phosphate (e.g., SKELITE™ manufactured by Biologix Inc.), thermoplastics such as polyaryletherketone (PAEK) including polyetheretherketone (PEEK), polyetherketoneketone (PEKK) and polyetherketone (PEK), carbon-PEEK composites, PEEK-BaSa polymeric rubbers, polyethylene terephthalate (PET), fabric, silicone, polyurethane, silicone-polyurethane copolymers, polymeric rubbers, polyolefin rubbers, hydrogels, semi-rigid and rigid materials, elastomers, rubbers, thermoplastic elastomers, thermoset elastomers, elastomeric composites, rigid polymers including polyphenylene, polyamide, polyimide, polyetherimide, polyethylene, epoxy, bone material including autograft, allograft, xenograft or transgenic cortical and/or corticocancellous bone, and tissue growth or differentiation factors, partially resorbable materials, such as, for example, composites of metals and calcium-based ceramics, composites of PEEK and calcium based ceramics, composites of PEEK with resorbable polymers, totally resorbable materials, such as, for example, calcium based ceramics such as calcium phosphate, tri-calcium phosphate (TOP), hydroxyapatite (HA)-TCP, calcium sulfate, or other resorbable polymers such as polyaetide, polyglycolide, polytyrosine carbonate, polycaroplaetohe and their combinations. Various components of spinal implant system 10 may have material composites, including the above materials, to achieve various desired characteristics such as strength, rigidity, elasticity, compliance, biomechanical performance, durability and radiolucency or imaging preference. The components of spinal implant system 10, individually or collectively, may also be fabricated from a heterogeneous material such as a combination of two or more of the above-described materials. The components of spinal implant system 10 may be monolithically formed, integrally connected or include fastening elements and/or instruments, as described herein.

Screw 12 includes a portion, such as, for example, a head 14 and a portion, such as, for example a shaft 16. Head 14 includes a tool engaging portion 18 configured to engage a surgical tool or instrument, as described herein. In one embodiment, portion 18 includes a hexagonal cross-section to facilitate engagement with a surgical tool or instrument, as described herein. In some embodiments, head 14 may have alternative cross-sections, such as, for example, rectangular, polygonal, hexalobe, oval, or irregular.

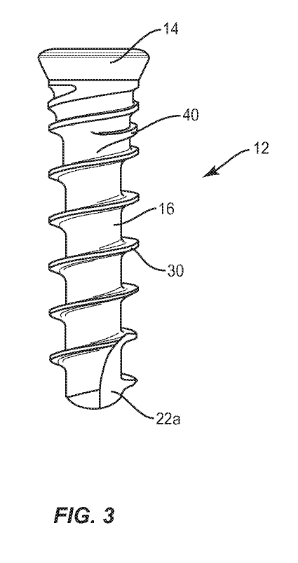

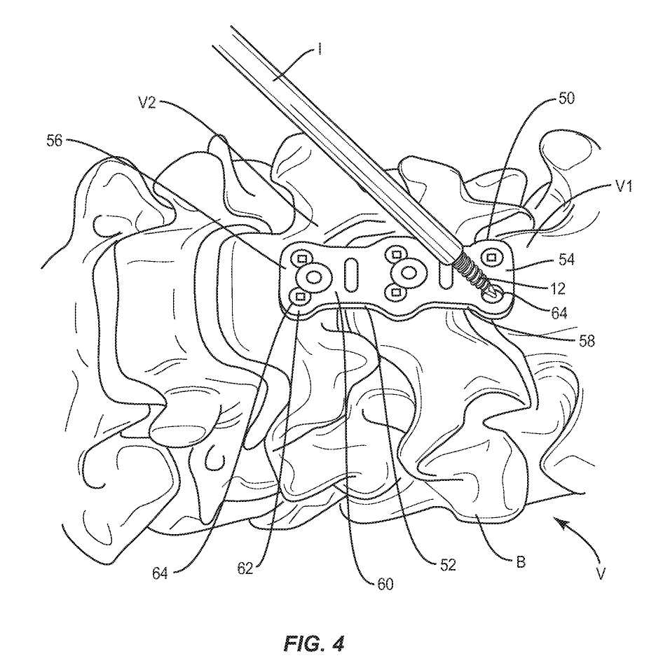

Shaft 16 extends along an axis X1. Shaft 16 includes an end 20 and an end 22. End 20 forms a section, such as, for example, a neck 24 with head 14. Neck 24 is disposed adjacent head 14. End 22 is configured to penetrate tissue, such as, for example, bone. In one embodiment, as shown in FIG. 1, end 22 includes a pointed tip configured to facilitate penetration of bone. In one embodiment, as shown in FIG. 3, end 22a includes a blunt tip configured to facilitate resist and/or prevent damage to surrounding tissue and/or nerves. In some embodiments, the blunt tip configuration can be employed to optimize bone purchase.

Shaft 16 includes an outer surface 26. Surface 26 includes a thread 30. Thread 30 extends along a length L1 of shaft 16 between end 20 and end 22. In one embodiment, thread 30 is continuous along surface 26. Thread 30 defines a pitch P1. In some embodiments, pitch P1 is a distance D1 from a crest 32 of a portion of thread 30 to an adjacent crest 32 of thread 30. In one embodiment, thread 30 includes a uniform pitch P1.

In one embodiment, thread 30 may include a single thread turn or a plurality of discrete threads. In some embodiments, other penetrating elements may be located on shaft 16, such as, for example, a nail configuration, barbs, expanding elements, raised elements, ribs, and/or spikes to facilitate engagement of shaft 16 with tissue. In some embodiments, thread 30 may be self-tapping or intermittent.

Neck 24 includes a resistance element, such as, for example, a thread 40. Thread 40 extends along neck 24 and within a space 34 between adjacent crests 32 of thread 30, as shown in FIG. 2. Thread 40 is separate and spaced apart from thread 30 along neck 24. In some embodiments, thread 40 extends in a parallel orientation relative to thread 30. In some embodiments, thread 40 may extend transverse, convergent, divergent, intersecting, staggered and/or offset relative to the thread 30.

In some embodiments, crests 32 may define a continuous space 34 along length Li and/or shaft 16. In some embodiments, crests 32 may define a plurality of separate cavities along length L1 and/or shaft 16. In one embodiment, thread 40 is oriented equidistant from adjacent crests 32. In some embodiments, thread 40 extends offset from a center point between adjacent crests 32.

Thread 40 defines a pitch P2. In some embodiments, pitch P2 is a distance D2 from a crest 33 of a portion of thread 40 to an adjacent crest 33 of thread 40. In one embodiment, thread 40 includes a uniform pitch P2. In one embodiment, pitch P2 is equal to pitch P1. In one embodiment, pitch P2 is less than pitch P1. In one embodiment, pitch P2 is greater than pitch P1. In some embodiments, screw 12 can include one or more resistance elements.

In some embodiments, the resistance element, such as, for example, thread 40 is configured to increase an insertion torque upon engagement of thread 40 with tissue, such as, for example, bone and/or a spinal implant, such as, for example, a cervical plate. The increase in torque generates and/or provides tactile indicia to a practitioner that bone screw 12 is approaching a fully seated position in bone and/or a spinal implant. In one embodiment, the resistance element may include a compressible washer disposed between head 14 and plate 50. In some embodiments, the washer includes a textured surface, such as, for example rough, arcuate, undulating, mesh, porous, semi-porous, dimpled and/or splined. In some embodiments, end 20 may include a resistance element, such as, for example, an increasing diameter thread 30, which may thread with a spinal implant, such as, for example, a surface of a plate. In some embodiments, end 20 may include a resistance element, such as, for example, an increasing minor diameter of thread 30 to cause increased resistance to insertion with tissue and/or a spinal implant.

In one embodiment, thread 40 engages tissue and/or a spinal implant to increase an insertion torque of bone screw 12 for generating and/or providing tactile indicia to a practitioner between a first and/or initial position and a second position, which may include approaching a fully seated position and/or a fully seated position with the tissue and/or the spinal implant. In some embodiments, the tactile indicia may comprise position indicia of bone screw 12 and/or engagement of thread 40 with tissue and/or a spinal implant may generate position indicia alone.

For example, thread 40 is engageable for contact with the bone and/or a cervical plate to generate tactile and/or position indicia to a practitioner for indicating a position of end 20 and/or end 22 relative to the bone and/or a cervical plate. In some embodiments, bone screw 12 is disposed in an initial position such that end 22 is disposed with bone and/or a cervical plate and end 20 is oriented a distance from the bone and/or a cervical plate.

Bone screw 12 translates distally into bone and/or a cervical plate such that thread 30 engages bone for fixation therewith. End 20 translates distally to engage bone and/or the cervical plate such that thread 40 engages bone and/or the cervical plate. In some embodiments, upon engagement of thread 40 with bone and/or the cervical plate, tactile and/or position indicia is generated indicating a second position of end 20 and/or end 22 relative to bone and/or the cervical plate indicating that bone screw 12 is approaching a fully-seated position.

In one embodiment, the spinal implant includes a cervical plate 50, as shown in FIG. 4. In some embodiments, cervical plate 50 includes an anterior cervical plate having a substantially rectangular configuration. In some embodiments, cervical plate 50 can be variously configured, such as, for example, tubular, oval, oblong, triangular, square, polygonal, irregular, uniform, non-uniform, variable, hollow and/or tapered. Cervical plate 50 includes a wall 52 extending between an end 54 and an end 56. In some embodiments, wall 52 can have alternate cross-section and/or thickness configurations, such as, arcuate, undulating, offset, staggered, tubular, oval, oblong, triangular, square, polygonal, irregular, uniform, variable, hollow and/or tapered.

Cervical plate 50 includes a surface 58 and a surface 60. Surface 58 includes substantially planar portions and is oriented in a first direction such that all or only a portion of surface 58 faces and/or engages tissue, as described herein. Surface 60 is oriented in a second direction, opposite to the first direction. In one embodiment, cervical plate 50 is configured for engagement with an anterior portion of vertebral tissue. In one embodiment, surface 58 has a frictional surface configuration for engagement with tissue to enhance fixation. In some embodiments, surface 58 may include alternate surface configurations, such as, for example, rough, arcuate, undulating, mesh, porous, semi-porous, dimpled and/or textured.

Cervical plate 50 includes a surface 62 that defines openings 64. Each opening 64 extends between surfaces 58, 60. Openings 64 are substantially circular and extend through the thickness of wall 52. In some embodiments, openings 64 can be variously configured, such as, for example, oval, oblong, triangular, square, polygonal, irregular, uniform, non-uniform and/or tapered. In some embodiments, plate 50 can have two or more openings 64.

In assembly, operation and use, spinal implant system 10, similar to the systems and methods described herein, includes bone screw 12 and is employed with a surgical procedure for treatment of a condition or injury of an affected section of the spine including vertebrae V. In one embodiment, as shown in FIG. 4, the components of spinal implant system 10 are attached to vertebrae V including vertebra V1 and vertebra V2.

In use, to treat a selected section of vertebrae V, a medical practitioner obtains access to a surgical site including vertebrae V in any appropriate manner, such as through incision and retraction of tissues. In some embodiments, spinal implant system 10 can be used in any existing surgical method or technique including open surgery, mini-open surgery, minimally invasive surgery and percutaneous surgical implantation, whereby vertebrae V is accessed through a mini-incision, or sleeve that provides a protected passageway to the area. Once access to the surgical site is obtained, the particular surgical procedure can be performed for treating the spine disorder.

An incision is made in the body of a patient and a cutting instrument (not shown) creates a surgical pathway in alignment with a surgical approach, as described herein, for implantation of components of spinal implant system 10. A preparation instrument (not shown) can be employed to prepare tissue surfaces of vertebrae V, as well as for aspiration and irrigation of a surgical region.

Cervical plate 50 is delivered along a surgical pathway to the surgical site. An instrument, such as, for example, an awl (not shown) is aligned and engaged with vertebrae V1, V2, to penetrate tissue and facilitate formation of pilot holes in vertebrae V1, V2, Head 14 is engaged with a surgical instrument, such as, for example, a driver I. Bone screw 12 is disposed in an initial position, as described herein, relative to bone B and cervical plate 50 at the surgical site.

Bone screw 12 is translated, as shown in FIG. 4, into bone B and opening 64 of cervical plate 50. Driver I is rotated causing screw 12 to translate axially within a pilot hole. Shaft 16 translates such that thread 30 engages bone B and/or the surface of opening 64. As bone screw 12 is translated into bone B and/or cervical plate 50, thread 30 engages bone B and/or opening 64 of cervical plate 50 with a constant torque applied to driver I.

As bone screw 12 translates and end 20 approaches bone B and cervical plate 50, thread 40 engages bone B and/or surface 60, for example, adjacent a substantially seated and/or fully seated position with bone B and cervical plate 50. Thread 40 generates tactile and/or position indicia, as described herein, causing an increase in torque required to translate bone screw 12 into bone B and cervical plate 50. The increase in required torque caused by the resistance and engagement of thread 40 with bone B and cervical plate 50 indicates positioning of end 20 and/or end 22 relative to bone B and/or surface 60 of cervical plate 50. In some embodiments, engagement of thread 40 with bone B and/or surface 60 indicates that bone screw 12 is approaching a fully-seated position and/or is disposed in a fully-seated position. In some embodiments, translation and/or further translation of bone screw 12 causes bone screw 12 to engage bone B and cervical plate 50 in a fully-seated position such that head 14 engages bone B and/or cervical plate 50 thereby preventing over-screwing and stripping of bone B and/or the surfaces of cervical plate 50.

Upon completion of a procedure, as described herein, the surgical instruments, assemblies and non-implanted components of spinal implant system 10 are removed and the incision(s) are closed. One or more of the components of spinal implant system 10 can be made of radiolucent materials such as polymers. Radiomarkers may be included for identification under x-ray, fluoroscopy, CT or other imaging techniques. In some embodiments, the use of surgical navigation, microsurgical and image guided technologies may be employed to access, view and repair spinal deterioration or damage, with the aid of spinal implant system 10. In some embodiments, system 10 may include one or a plurality of rods, plates, connectors and/or bone fasteners for use with a single vertebral level or a plurality of vertebral levels.

In some embodiments, one or more bone fasteners, such as, for example, bone screw 12 may be engaged with tissue in various orientations, such as, for example, series, parallel, offset, staggered and/or alternate vertebral levels. In some embodiments, one or more of fasteners may comprise multi-axial screws, sagittal angulation screws, pedicle screws, mono-axial screws, uni-planar screws, facet screws, fixed screws, tissue penetrating screws, conventional screws, expanding screws, wedges, anchors, buttons, cups, snaps, friction fittings, compressive fittings, expanding rivets, staples, nails, adhesives, posts, fixation plates and/or posts.

In one embodiment, spinal implant system 10 includes an agent, which may be disposed, packed, coated or layered within, on or about the components and/or surfaces of spinal implant system 10. In some embodiments, the agent may include bone growth promoting material, such as, for example, bone graft to enhance fixation of the components and/or surfaces of spinal implant system 10 with vertebrae. In some embodiments, the agent may include one or a plurality of therapeutic agents and/or pharmacological agents for release, including sustained release, to treat, for example, pain, inflammation and degeneration.

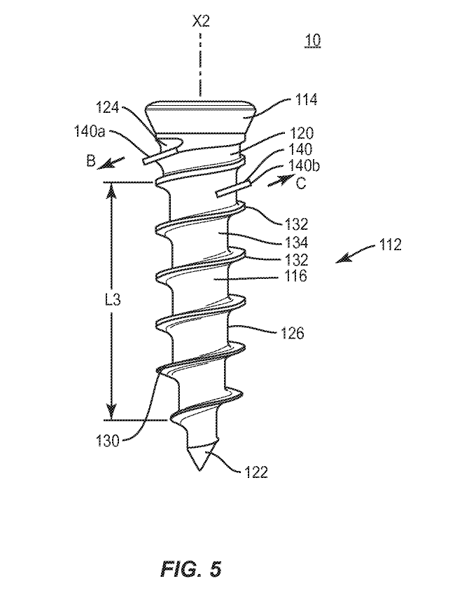

In one embodiment, as shown in FIG. 5, spinal implant system 10, similar to the systems and methods described herein, includes a bone screw 112, similar to bone screw 12. Screw 112 includes a head 114 and a shaft 116. Head 114 includes a tool engaging portion 118 configured to engage a surgical tool or instrument, as described herein. Shaft 116 extends along an axis X2. Shaft 116 includes an end 120 and an end 122. End 120 forms a neck 124 with head 114. Neck 124 is disposed adjacent head 114. End 122 is configured to penetrate tissue, such as, for example, bone.

Shaft 116 includes an outer surface 126. Surface 126 includes a thread 130, similar to thread 30 described herein. Thread 130 extends along a length L3 of shaft 116 between end 120 and end 122. Thread 130 is continuous along surface 126. Thread 130 defines a uniform pitch that is a distance from a crest 132 of a portion of thread 130 to an adjacent crest 132 of thread 130. Thread 130 defines a space 134 between adjacent crests 132 of thread 130.

Neck 124 includes a resistance element, such as, for example, a penetrating element 140. Penetrating element 140 extends from neck 124 and/or from space 134. In one embodiment, penetrating element 140 includes, such as, for example, spikes 140a, 140b. In one embodiment, a spike 140a protrudes in a first direction transverse to axis X2, as shown by arrow B in FIG. 5. In one embodiment, a spike 140b protrudes in a second direction transverse to axis X2, as shown by arrow C in FIG. 5. In some embodiments, penetrating element 140 may include, such as, for example, a nail configuration, barbs, expanding elements, raised elements, ribs, bosses and/or protrusions. In some embodiments, bone screw 112 can include one or more penetrating elements.

In some embodiments, spikes 140a, 140b are configured to increase an insertion torque upon engagement of spikes 140a, 140b with tissue and/or a spinal implant, as described herein. The increase in torque generates and/or provides tactile indicia to a practitioner that bone screw 112 is approaching a fully seated position in bone and/or spinal implant. In one embodiment, spikes 140a, 140b engage tissue and/or a spinal implant to increase an insertion torque of bone screw 112 for generating and/or providing tactile indicia to a practitioner between a first and/or initial position and a second position, which may include approaching a fully seated position and/or a fully seated position with the tissue and/or the spinal implant. In some embodiments, the tactile indicia may comprise position indicia of bone screw 112 and/or engagement of spikes 140a, 140b with tissue and/or a spinal implant may generate position indicia alone.

It will be understood that various modifications may be made to the embodiments disclosed herein. Therefore, the above description should not be construed as limiting, but merely as exemplification of the various embodiments. Those skilled in the art will envision other modifications within the scope and spirit of the claims appended hereto.

What is claimed is:

1. A spinal implant comprising:
a proximal end including a thread and at least one resistance element; and
a distal end including the thread and being configured to penetrate tissue,
wherein the at least one resistance element is engageable to increase a resistance during penetration of the tissue.

2. A spinal implant as recited in claim 1, wherein the at least one resistance element engages the tissue and generates tactile indicia.

1. l implant as recited in claim 1, wherein the at least one resistance element engages tissue comprising bone to generate tactile indicia and increase required insertion torque of the spinal implant with the bone.

4. A spinal implant as recited in claim 1, wherein the at least one resistance element engages tissue to generate positioning indicia of the position of at least one of the ends relative to the tissue.

5. A spinal implant as recited in claim 1, wherein the at least one resistance element engages tissue including bone to generate tactile indicia and positioning indicia of the proximal end relative to the bone.

6. A spinal implant as recited in claim 1, wherein the thread includes a first thread and the at least one resistance element includes a second thread spaced apart from the first thread.

7. A spinal implant as recited in claim 6, wherein the second thread has a pitch equal to a pitch of the first thread.

8. A spinal implant as recited in claim 6, wherein the second thread has a pitch different from a pitch of the first thread.

9. A spinal implant as recited in claim 6, wherein the second thread engages the tissue and generates tactile indicia.

10. A spinal implant as recited in claim 1, wherein the distal end includes a self-tapping tip.

11. A spinal implant as recited in claim 1, wherein the distal end includes a blunt tip.

12. A spinal implant as recited in claim 1, wherein the distal end includes a pointed tip.

13. A spinal implant as recited in claim 1, wherein the at least one resistance element includes at least one penetrating element.

14. A spinal implant as recited in claim 13, wherein the at least one penetrating element includes a first penetrating element oriented in a first direction and a second penetrating element oriented in a second direction.

15. A spinal implant as recited in claim 1, wherein a bone fastener includes the ends, and further comprising a plate engageable with the bone fastener.

16. A spinal implant as recited in claim 15, wherein at least one resistance element engages the tissue and/or the plate to generate positioning indicia of at least one of the ends relative to the plate.

17. A spinal implant as recited in claim 15, wherein the at least one resistance element engages tissue including bone to generate tactile indicia and positioning indicia of the proximal end relative to the plate.

18. A spinal implant comprising:
a proximal end including a first thread and a second thread spaced apart from the first thread; and
a distal end including the first thread and being configured to penetrate tissue,
wherein the second thread engages tissue comprising bone to generate tactile indicia and increase required insertion torque of the spinal implant with the bone and generate positioning indicia of the proximal end relative to the bone.

19. A spinal implant as recited in claim 18, wherein the second thread has a pitch equal to a pitch of the first thread.

20. A spinal implant system comprising:
a bone fastener having a proximal end including a thread and at least one resistance element and a distal end including the thread and being configured to penetrate tissue; and
a plate engageable with the bone fastener,
wherein the at least one resistance element engages the tissue and/or the plate to generate tactile indicia and/or positioning indicia of at least one of the ends relative to the plate.
